(19) United States
(12) Patent Application Publication
(10) Pub. No.: US 2024/0277254 A1
(43) Pub. Date: Aug. 22, 2024

(54) SYSTEMS AND METHODS FOR DETECTION OF POSITIVE END-EXPIRATORY PRESSURE (PEEP) USING DIAPHRAGMATIC ULTRASOUND

(71) Applicant: KONINKLIJKE PHILIPS N.V., EINDHOVEN (NL)

(72) Inventors: Jaap Roger Haartsen, Eindhoven (NL); Roberto Buizza, Eindhoven (NL); Joerg Sabczynski, Hamburg (DE); Rafael Wiemker, Hamburg (DE); Thomas Koehler, Hamburg (DE); Cornelis Petrus Hendriks, Eindhoven (NL); Michael Polkey, London (GB); Bernard Hete, Pittsburgh, PA (US)

(21) Appl. No.: 18/434,965

(22) Filed: Feb. 7, 2024

Related U.S. Application Data
(60) Provisional application No. 63/444,353, filed on Feb. 9, 2023.

(51) Int. Cl. A61B 5/087; A61B 5/00; A61B 8/08; A61M 16/00; A61M 16/10

(52) U.S. Cl. CPC A61B 5/087; A61B 5/742; A61B 8/08; A61B 8/5292; A61M 16/022; A61M 16/10

(57) ABSTRACT

A respiration monitoring device includes at least one electronic processor programmed to perform a respiration monitoring method including receiving ultrasound imaging data of a diaphragm of a patient as a function of time during inspiration and expiration while the patient undergoes mechanical ventilation therapy with a mechanical ventilator; receiving respiratory data of the patient as a function of time during the inspiration and expiration while the patient undergoes the mechanical ventilation therapy; calculating an intrinsic positive end-expiratory pressure (iPEEP) value for the patient based on the ultrasound imaging data and the respiratory data; and displaying, on a display device, a representation of the calculated iPEEP value.

### CROSS-REFERENCE TO RELATED APPLICATIONS

This patent application claims the priority benefit under 35 U.S.C. § 119(c) of U.S. Provisional Application No. 63/444,353, filed on Feb. 9, 2023, the contents of which are herein incorporated by reference.

The following relates generally to the respiratory therapy arts, mechanical ventilation arts, ventilator induced lung injury (VILI) prevention arts, and related arts.

### BACKGROUND

Intrinsic positive end-expiratory pressure (PEEP), also known as autoPEEP or iPEEP, occurs when the expiratory time is shorter than the time needed to fully deflate the lungs to its functional residual capacity (FRC), preventing the lung and chest wall from reaching an elastic equilibrium point. This is sometimes referred to as ‘gas trapping.’ When this problem occurs, a portion of each subsequent tidal volume may be retained in the patient's lungs, a phenomenon sometimes referred to as breath stacking or dynamic hyperinflation, resulting in increased end-expiratory pressure in the alveoli. If this goes unrecognized, depending on the ventilation mode, it may result in barotrauma, volutrauma, tidal volume reduction, hypotension, patient-ventilator asynchrony, or death. The main pathophysiological factors involved are insufficient time for emptying the lung and/or expiratory flow limitation (EFL), e.g., due to dynamic small airway collapse due to pressure from hyperinflated lung units, mainly for chronic obstructive pulmonary disease (COPD).

Diaphragmatic ultrasonography (US) allows for quantification of diaphragm thickness, strain (rate) and excursion, and with this also the respiratory rate and duration of each contraction. Diaphragm thickness (expressed as thickening fraction) and strain reflect contractile activity and correlate well with diaphragmatic electrical activity and transdiaphragmatic pressure. Consequently, thickness and strain may be used as a surrogate for respiratory effort. Applications of diaphragmatic ultrasound include assessment of diaphragm function, atrophy detection, weaning prediction, and mechanical ventilation (MV) setting management.

Ultrasound (US) measurements of diaphragm thickness are typically quantified in terms of a diaphragm thickening fraction (TFdi or TFDI). Diaphragm thickening fraction measurements are carried out by an operator who looks at the patient and takes an ultrasound image at end inhalation and end exhalation. With suitable positioning of the ultrasound probe, typically in an intercostal position, the superior and inferior boundaries or surfaces of the diaphragm appear as contrast lines in the ultrasound image, and the diaphragm thickness is then the separation of these two contrast lines in the image (possibly with suitable correction of angular position of the ultrasound beam respective to the surface normal of the diaphragm plane). The diaphragm thickness fraction is then determined by subtracting the diaphragm thickness Tₑᵢ measured at end inhalation from the diaphragm thickness Tₑₑ measured at end exhalation, and dividing the difference by the diaphragm thickness Tₑₑ at end-exhalation according to Equation 1:

$$TFdi = \frac{{T}_{ei - {T}_{ee}}}{{T}_{e e}} * 100 \% \qquad ( 1 )$$

with TFdi being the diaphragm thickness fraction.

Presence of intrinsic PEEP is detected by automatic or manual inspection of flow curves (i.e., by checking whether the expiratory flow is still occurring just before start of a new inhalation). The value of the intrinsic PEEP could be obtained from an expiratory hold maneuver (i.e. by forcing the expiratory flow to zero), leading to an equilibration of alveoli and airway pressure, and measuring the airway pressure (equal to intrinsic PEEP). This works well for a passive patient during controlled mechanical ventilation.

However, for an actively breathing patient during spontaneous or assisted ventilation the presence of intrinsic PEEP causes the inspiratory effort to start during the expiration phase to overcome the intrinsic PEEP before triggering a new breath. Consequently, it is difficult to manually or automatically detect it by inspection of pressure and flow curves. Also, a reliable measurement of the intrinsic PEEP value is much more difficult in the presence of respiratory muscle activity since during an expiratory hold maneuver it is not possible to determine which amount of measured positive airway occlusion pressure is due to expiratory muscle activity. In this case one feasible method is to measure the drop in esophageal pressure (as a surrogate for the pleural pressure) just before start of inspiration, and subsequently subtract the part due to respiratory muscle activity determined from esophageal pressure. However, this method is not routinely used because of its invasive nature, the need for a dedicated ventilator system supporting esophageal pressure measurements and the need for time-consuming calibration steps.

The following discloses certain improvements to overcome these problems and others.

### SUMMARY

In one aspect, a respiration monitoring device includes at least one electronic processor programmed to perform a respiration monitoring method including receiving ultrasound imaging data of a diaphragm of a patient as a function of time during inspiration and expiration while the patient undergoes mechanical ventilation therapy with a mechanical ventilator; receiving respiratory data of the patient as a function of time during the inspiration and expiration while the patient undergoes the mechanical ventilation therapy; calculating an intrinsic positive end-expiratory pressure (iPEEP) value for the patient based on the ultrasound imaging data and the respiratory data; and displaying, on a display device, a representation of the calculated iPEEP value.

In another aspect, a respiration monitoring method includes, with an electronic controller, receiving ultrasound imaging data of a diaphragm of a patient as a function of time during inspiration and expiration while the patient undergoes mechanical ventilation therapy with a mechanical ventilator; receiving respiratory data of the patient as a function of time during the inspiration and expiration while the patient undergoes the mechanical ventilation therapy; calculating a positive end-expiratory pressure (PEEP) value for the patient based on the ultrasound imaging data and the respiratory data; and displaying, on a display device, a representation of the calculated PEEP value.

One advantage resides in noninvasively determining the intrinsic PEEP value for a mechanically ventilated but actively breathing patient.

Another advantage resides in adjusting mechanical ventilator operation to compensate for a non-zero intrinsic PEEP.

Another advantage resides in using a combination of ultrasound imaging data and at least one ventilator waveform to determine an intrinsic PEEP value for a mechanical ventilated patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative respiration monitoring device in accordance with the present disclosure.

FIG. 2 shows an example flow chart of operations suitably performed by the system of FIG. 1.

FIG. 3 shows an example in which the onset of inhalation effort by the patient occurs before the mechanical ventilator begins to apply inhalation pressure.

FIG. 4 shows an example flow chart of detection of an intrinsic PEEP value.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

The following discloses using diaphragmatic ultrasound to detect and measure an intrinsic PEEP (iPEEP) value in an actively breathing patient undergoing mechanical ventilation therapy. Diaphragmatic ultrasound is a non-invasive imaging modality that is readily available in the ICU which is used for visualization of the diaphragm. Diaphragmatic ultrasound allows a clinician to determine the thickness and thickness fraction of the diaphragm from which respiratory activity and hence the onset of inspiration can be derived to determine the iPEEP value.

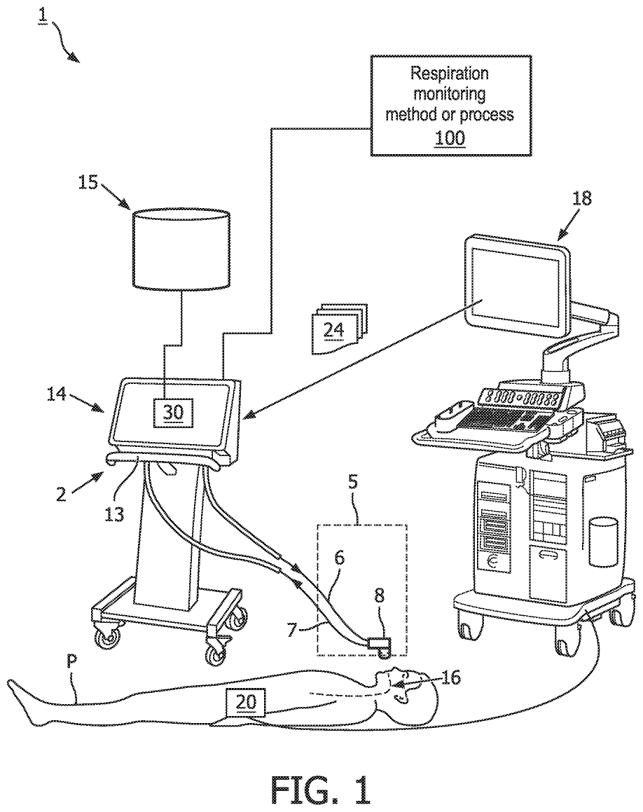

With reference to FIG. 1, a diaphragm measurement device 1 is shown. A mechanical ventilator 2 is configured to provide ventilation therapy to an associated patient P is shown. As shown in FIG. 1, the mechanical ventilator 2 is connected with a patient breathing circuit 5 to deliver mechanical ventilation to the patient P. The patient breathing circuit 5 includes typical components for a mechanical ventilator, such as an inlet line 6 (also called inhalation limb 6), an optional outlet line 7 (also called exhalation limb 7; this may be omitted if the ventilator employs a single-limb patient circuit), a connector or port 8 for connecting with an endotracheal tube (ETT) 16, or a mask or other patient interface, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the inhalation limb 6 of the patient breathing circuit 5 at a programmed pressure and/or flow rate to ventilate the patient via an ETT, and optionally also handling exhaled air received at the mechanical ventilator 2 via the exhalation limb 7 of the patient breathing circuit 5. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (most of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 also shows a medical imaging device 18 (also referred to as an image acquisition device, imaging device, and so forth). As primarily described herein, the medical imaging device 18 comprises an ultrasound (US) medical imaging device 18. The illustrative embodiments employ brightness mode (B-mode) ultrasound imaging to assess the diaphragm thickness metric. However, other types of ultrasound imaging or data are contemplated, such as motion mode (M-mode) data collected as a single ultrasound line over a time interval, A-mode, or so forth.

In some examples, the medical imaging device 18 includes an ultrasound probe 20 configured to acquire US imaging data (i.e., US images) 24 of the diaphragm of the patient P. In a more particular example, the medical imaging device 18 includes an ultrasound patch 20 that is wearable by the patient P (e.g., on the abdomen or chest of the patient P in position to image the diaphragm of the patient, as shown in FIG. 1). The US patch 20 is positioned to acquire the US images 24 of the diaphragm of the patient P. For example, the US patch 20 is configured to acquire imaging data of a diaphragm of the patient P, and more particularly US imaging data related to a thickness of the diaphragm of a patient P during inspiration and expiration while the patient P undergoes mechanical ventilation therapy with the mechanical ventilator 2. The electronic processor 13 controls the ultrasound imaging device 18 to receive the ultrasound imaging data 24 of the diaphragm of the patient P from the US patch 20. Although only one US patch 20 is shown in FIG. 1, it will be appreciated that any suitable number of patches can be attached to the patient P. The ultrasound patch(es) 20 allow for continuous and automatic determination of the diaphragm thickness data (Tdi) from the acquired ultrasound imaging data 24.

The non-transitory computer readable medium 15 stores instructions executable by the electronic controller 13 to perform a respiration monitoring method or process 100.

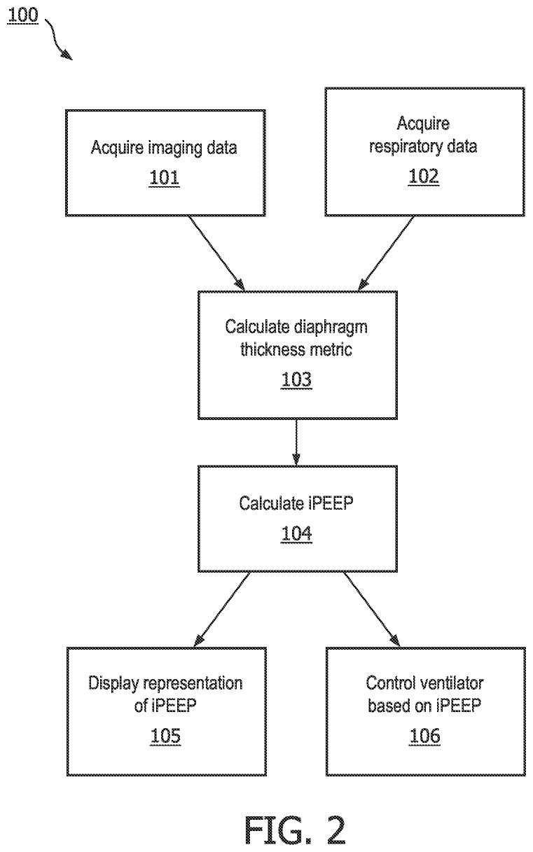

With reference to FIG. 2, and with continuing reference to FIG. 1, an illustrative embodiment of the respiration monitoring method 100 is diagrammatically shown as a flowchart. At an operation 101, the US imaging data 24 of the diaphragm of the patient P is received as a function of time during inspiration and expiration while the patient undergoes mechanical ventilation therapy with the mechanical ventilator 2. To do so, the electronic controller 13 can control the ultrasound patch 20 to acquire the ultrasound imaging data 24 and receive the ultrasound imaging data 24 of the diaphragm of the patient P from the ultrasound patch 20 and/or the medical imaging device 18.

At an operation 102, respiratory data of the patient P during inspiration and expiration is received as a function of time while the patient undergoes the mechanical ventilation therapy with the mechanical ventilator 2. Typically, the operations 101 and 102 are performed concurrently, that is, over the same time interval. The respiratory data of the patient P can include one or more of an airway pressure in an airway of the patient P or an airway flow in an airway of the patient P measured during ventilation therapy by the mechanical ventilator 2. Notably, airway pressure and airway flow are commonly monitored during mechanical ventilation, so this data is typically available for any mechanically ventilated patient.

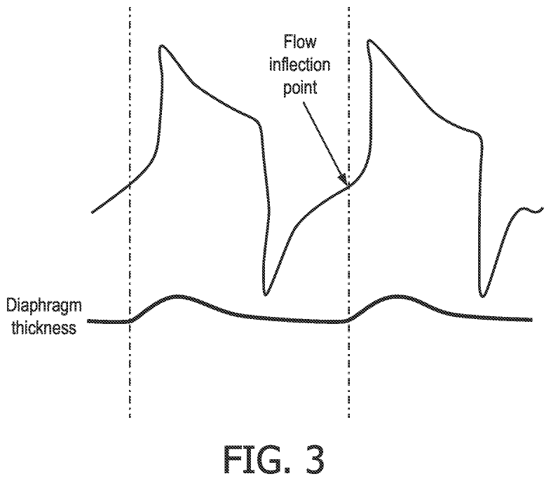

At an operation 103, a diaphragm thickness metric as a function of time is calculated based on the received US imaging data 24 of the diaphragm of the patient P. As previously noted, with suitable positioning of the ultrasound probe in the operation 101, typically in an intercostal position, the superior and inferior boundaries or surfaces of the diaphragm appear as contrast lines in the ultrasound image, and the diaphragm thickness is then measured as the separation of these two contrast lines in the image (possibly with suitable correction of angular position of the ultrasound beam respective to the surface normal of the diaphragm plane). However, unlike the case for determining the diaphragm thickening fraction or ratio (see Equation 1), for the iPEEP measurements disclosed herein the operation 103 determines diaphragm thickness as a function of time, with sufficient temporal resolution to detect onset of the inspiratory effort by the patient P as an onset of increased diaphragm thickness. This is seen in the lower curve of FIG. 3, which shows the onset of inspiratory effort (marked as a dashed vertical line in FIG. 3). The onset of inspiratory effort corresponds to a time of onset of an increase in the diaphragm thickness as the patient's inspiratory effort is executed by contraction of the diaphragm and its resultant thickening.

At an operation 104, a iPEEP value for the patient P is calculated based on the ultrasound imaging data 24 and the respiratory data. For example, the iPEEP value is calculated based on the diaphragm thickness metric (from the diaphragm thickness metric calculation operation 103) as a function of time and the respiratory data. To do so, a time corresponding to start of inhalation is identified based on (i) the determined diaphragm thickness metric, (ii) the received respiratory data, or (iii) a combination of both. In one example, the iPEEP value is calculated as iPEEP=−R\dot{V}(t) where R is a respiratory resistance of lungs of the patient and V(t) is an airflow at the identified time corresponding to start of inhalation which is part of the received respiratory data of the patient. In another example, an updated iPEEP value applied by the mechanical ventilator 2 can be determined from the received respiratory data 24, and a total PEEP value can be calculated as a sum of the calculated iPEEP value and the determined updated iPEEP value.

In some embodiments, the received respiratory data of the patient P as a function of time includes a ventilator flow waveform generated during the mechanical ventilation therapy. In this embodiment, the iPEEP calculation operation 104 include (i) detecting an onset of inspiratory effort by the patient P based on the ultrasound imaging data 24, (ii) determining an air flow value from the lungs of the patient P at the detected onset of inspiratory effort by the patient P from the ventilator flow waveform, and (iii) calculating the iPEEP value based on the determined air flow value from the lungs of the patient P at the detected onset of inspiratory effort by the patient P. In some embodiments, the mechanical ventilator 2 is triggered to initiate a breath based on the determined onset of inspiratory effort by the patient P.

In a particular example, referring now to FIG. 3, the onset of inspiratory effort is detected from ultrasound based diaphragmatic thickness measurements (i.e., at the start of the diaphragm thickening). Next, a corresponding air flow value is determined from a ventilator flow waveform shown in FIG. 3. If the air flow is non-zero at this point, this is a sign of presence of iPEEP. Another indication of the presence of iPEEP is the time delay between the flow inflection point and the onset of the patient's inspiratory effort. This is because it takes time for the diaphragm (and other respiratory muscles) to first counterbalance the iPEEP for this effort to generate a small pressure drop (i.e., in the presence of a closed circuit) or to initiate the inspiratory flow (i.e., in an open circuit). The mechanical ventilator 2 and the ultrasound imaging device 18 need to be synchronized for accurate results, for example the respiratory muscle onset detected from the diaphragmatic ultrasound can be used to trigger the next breath when the ventilator and ultrasound machine are synchronized (see, e.g., U.S. Provisional App. No. 63/426,069, filed Nov. 17, 2022).

FIG. 3 shows an example of a ventilator flow waveform, which can be displayed on the display device 14. A flow waveform (shown at the top of FIG. 3) and a diaphragm thickness waveform (shown at the bottom of FIG. 3) are shown. A dashed line indicates the onset of inspiratory effort. The onset of diaphragm thickening starts during the expiration phase since the intrinsic PEEP needs to be overcome first. In additional embodiments also the pressure waveforms can be displayed.

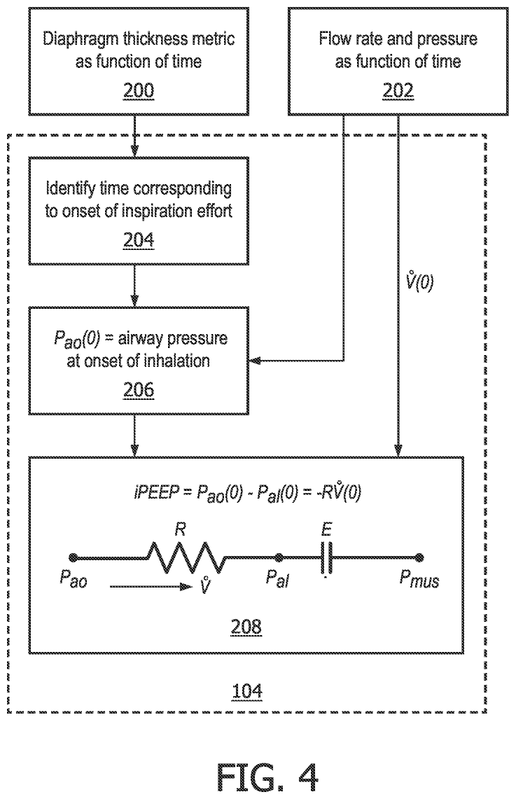

With reference to FIG. 4, an example of one implementation of the iPEEP calculation 104 of FIG. 2 is shown by way of a flowchart. The data input are the diaphragm thickness metric as a function of time 200 determined as described above, and the respiratory data comprising the flow rate V and airway pressure P as a function of time 202 both of which are routinely measured during mechanical ventilation. In an operation 204, a time t=0 corresponding to the onset of inspiration effort is detected, for example by identifying the inflection point in the diaphragm thickness metric as a function of time 200 indicated by the vertical dashed lines in FIG. 3. In an operation 206, the airway pressure Pₐₒ (0) at the time t=0 corresponding to onset of inspiration effort is obtained from the respiratory data 202.

Next, in an operation 208 the iPEEP value is determined. iPEEP is the pressure difference between the airway (Pₐₒ) and the alveoli (Pₐₗ) just before start of inhalation (t=0). From a single compartment model indicated in the block 208 of FIG. 4, it can be derived that this equals the product of respiratory resistance R and flow (\dot{V}) according to Equation 1:

$$iPEEP = {P}_{ao} ( 0 ) - {P}_{al} ( 0 ) = - R \dot{V} ( 0 ) 7 \qquad ( 1 )$$

Respiratory resistance values can be determined as disclosed in, for example, U.S. Provisional App. No. 63/407,772, filed Sep. 19, 2022

Once the iPEEP value is known, it can be suggested to increase the PEEP setting of a quantity equal to the iPEEP. This “total” PEEP can be suggested as a new setpoint for a new PEEP value to eliminate iPEEP (and hence enabling the lung to completely empty because of the larger driving pressure). Also, from this iPEEP value, an exhalation time could be estimated and proposed for complete emptying of the lung. Other proposed actions may include reduction of the tidal volume and breathing rate. It should be noted that instead of suggesting above actions to the caregiver, these actions can also be automatically implemented and controlled in a closed-loop system. The onset of the respiratory muscle to determine the iPEEP could be obtained also with a surface EMG synchronized with the ventilator

Referring back to FIG. 2, at an operation 105, a representation 30 of the calculated PEEP value is displayed on the display device 14. In some examples, when multiple ultrasound patches 20 are attached to different locations on the chest of the patient P, the representation 30 can include a color map showing distribution of activity of the diaphragm.

In some embodiments, at an operation 106, the mechanical ventilator 2 can be controlled to adjust one or more parameters of the mechanical ventilation therapy delivered to the patient based on the calculated diaphragm thickness metric. For example, an adjustment of at least one mechanical ventilation setting of the mechanical ventilator 2 can be determined based on the calculated PEEP value. The determined adjustment can be applied to the mechanical ventilator 2 to adjust the mechanical ventilation therapy to the patient P. The determined adjustment can be displayed on the display device 14 as a proposed adjustment to adjust the mechanical ventilation therapy to the patient P. The determined adjustment can be, for example, a change in an exhalation time, a change in a pressure applied by the mechanical ventilator 2 during exhalation, and so forth.

In some embodiments, instead of calculating the diaphragm thickness metric at the operation 103, a negative pressure value can be determined based on respiratory effort by the patient P. In response to the calculated iPEEP value being above a threshold, a modified configuration of the mechanical ventilator 2 effective to apply a negative pressure to the patient during exhalation (or inferior to the PEEP settings) by the patient P during the mechanical ventilation therapy can be determined to assist with exhalation by the patient P. The determined modified configuration can be displayed on the display device 14, or the mechanical ventilator 2 can be controlled to implement the determined modified configuration.

In this embodiment, the negative pressure is applied on an exhalation limb 7 of the patient breathing circuit 5 (see FIG. 1) during the exhalation phase to help the exhalation by generating a larger pressure differential between the alveoli and the airways (i.e. to enable a faster exhalation). Viewed another way, the negative pressure applied to the exhalation limb 7 operates to actively draw air out of the lungs of the patient P, thus removing the residual air from the lungs that otherwise results in the undesirable positive value of iPEEP. The value of the negative pressure could be calculated from the intrinsic PEEP value as determined using a model. The negative pressure value could be suggested to the caregiver or automatically implemented and controlled in a closed-loop system. In cases of expiratory flow limitation, a reduction of the airway pressure during the exhalation phase may not result in a reduction of the flow before start of a new inhalation. However, monitoring the changes in the flow just before start of a new inhalation upon applied changes in airway pressure during the exhalation phase allows to detect presence of expiratory flow limitation. This could be indicated to the caregiver. If expiratory flow limitation is detected, a higher PEEP value can be suggested or automatically implemented using a closed-loop solution.

In some embodiments, instead of calculating the diaphragm thickness metric at the operation 103, a diaphragmatic excursion value of the diaphragm can instead be determined for successive breaths based on the ultrasound imaging data 24 as a function of time. An alert 30 can be displayed on the display device 14 in response to an increase or decrease of the diaphragmatic excursion values for the successive breaths over time satisfying an alert criterion.

In this embodiment, with ultrasound, it is possible to measure the excursion of the diaphragm. Provided the excursion can be measured in an absolute manner, a gradual shift would indicate an increasing expansion of the lungs due to dynamic hyperinflation. This provides an additional (i.e., indirect) manner to detect intrinsic PEEP with ultrasound. The advantage is that it allows to directly measure the cumulative effect of the intrinsic PEEP, instead of integrating a flow curve. This information is meaningful because the expansion of the chest activates receptors which causes arousals and discomfort. Additionally, an expansion of the lungs increases the risk of overstretching and VILI.

In some embodiments, the PEEP calculation operation 104 can include determining lung sliding of lungs of the patient for successive breaths based on speckle tracking performed on the ultrasound imaging data 24 as a function of time. An alert 30 can be displayed on the display device 14 in response to an increase or decrease of the lung sliding for the successive breaths over time satisfying an alert criterion.

In this embodiment, while the excursion of the diaphragm provides direct information on the lung volume and thus on a progressively increasing end-expiratory volume due to intrinsic PEEP, indirect information is also available from the pleura. Lung Ultrasound with speckle tracking allows quantifying lung sliding. It is expected that with progressively increasing lung volume the speckle position in the end-expiratory breathing phase is moving caudally while at the same time the distance between speckles is increasing. This effect can be small from breath to breath, but it could be detected over several breathing cycles and could thus trigger a warning. This embodiment would measure the cumulative effect of intrinsic PEEP.

In some embodiments, the electronic controller 13 is programmed to control the mechanical ventilator 2 having an inhalation limb and an exhalation limb to perform mechanical ventilation of a patient; and during the mechanical ventilation, in response to determining an iPEEP is higher than a threshold adjust the mechanical ventilation of the patient to apply a negative pressure on the exhalation limb during an exhalation phase of the mechanical ventilation of the patient.

In some embodiments, the electronic controller 13 is programmed to determine a diaphragmatic excursion value of a diaphragm of a patient undergoing mechanical ventilation for successive breaths based on ultrasound imaging data of the diaphragm as a function of time; and display, on the display device 14, an alert in response to an increase or decrease of the diaphragmatic excursion values for the successive breaths over time satisfying an alert criterion.

In some embodiments, the electronic controller 13 is programmed to determine lung sliding of lungs of a patient undergoing mechanical ventilation for successive breaths based on ultrasound imaging data of the lungs of the patient as a function of time; and display, on the display device 14, an alert in response to an increase or decrease of the lung sliding for the successive breaths over time satisfying an alert criterion.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A respiration monitoring device, comprising at least one electronic processor programmed to perform a respiration monitoring method including:
receiving ultrasound imaging data of a diaphragm of a patient as a function of time during inspiration and expiration while the patient undergoes mechanical ventilation therapy with a mechanical ventilator;
receiving respiratory data of the patient as a function of time during the inspiration and expiration while the patient undergoes the mechanical ventilation therapy;
calculating an intrinsic positive end-expiratory pressure (iPEEP) value for the patient based on the ultrasound imaging data and the respiratory data; and
displaying, on a display device, a representation of the calculated iPEEP value.

2. The device of claim 1, wherein the method further includes:
determining a diaphragm thickness metric as a function of time based on the received ultrasound imaging data of the diaphragm of the patient;
wherein the iPEEP value is calculated based on the diaphragm thickness metric as a function of time and the respiratory data.

3. The device of claim 2, wherein the calculating of the iPEEP value includes:
identifying a time corresponding to start of inhalation based on the determined diaphragm thickness metric; and
calculating the iPEEP value at the identified time corresponding to start of inhalation.

4. The device of claim 2, wherein the calculating of the iPEEP value includes:
identifying a time corresponding to start of inhalation based on the received respiratory data; and
calculating the iPEEP value at the identified time corresponding to start of inhalation.

5. The device of claim 3, wherein the iPEEP value is calculated as iPEEP=−R\dot{V}(t) where R is a respiratory resistance of lungs of the patient and \dot{V}(t) is an airflow at the identified time corresponding to start of inhalation which is part of the received respiratory data of the patient.

6. The device of claim 1, wherein the received respiratory data of the patient as a function of time includes a ventilator flow waveform generated during the mechanical ventilation therapy, and calculating the iPEEP value comprises:
detecting an onset of inspiratory effort by the patient based on the ultrasound imaging data;
determining an air flow value from the lungs of the patient at the detected onset of inspiratory effort by the patient from the ventilator flow waveform; and
calculating the iPEEP value based on the determined air flow value from the lungs of the patient at the detected onset of inspiratory effort by the patient.

7. The device of claim 6, wherein the method further includes:
triggering the mechanical ventilator to initiate a breath based on the determined onset of inspiratory effort by the patient.

8. The device of claim 1, wherein the method further includes:
determining a diaphragmatic excursion value of the diaphragm for successive breaths based on the ultrasound imaging data as a function of time; and
displaying, on the display device, an alert in response to an increase or decrease of the diaphragmatic excursion values for the successive breaths over time satisfying an alert criterion.

9. The device of claim 2, wherein calculating the iPEEP value from the calculated diaphragm thickness metric comprises:
determining lung sliding of lungs of the patient for successive breaths based on speckle tracking performed on the ultrasound imaging data as a function of time; and
displaying, on the display device, an alert in response to an increase or decrease of the lung sliding for the successive breaths over time satisfying an alert criterion.

10. The device of claim 1, wherein the method further includes:
determining a negative pressure value based on a respiratory effort of the patient; and
in response to the calculated iPEEP value being above a threshold, determining a modified configuration of the mechanical ventilator effective to apply a negative pressure to the patient during exhalation by the patient during the mechanical ventilation therapy to assist with exhalation by the patient; and
one of (i) displaying, on the display device, the determined modified configuration, or (ii) controlling the mechanical ventilator to implement the determined modified configuration.

11. The device of claim 1, wherein the method further includes:
determining an updated iPEEP value applied by the mechanical ventilator from the received respiratory data;
calculating a total PEEP value as a sum of the calculated iPEEP value and the determined updated iPEEP value.

12. The device of claim 1, wherein the method further includes:
determining an adjustment of at least one mechanical ventilation setting of the mechanical ventilator based on the calculated iPEEP value; and
one of: (i) applying the determined adjustment to the mechanical ventilator or (ii) displaying, on the display device, the determined adjustment as a proposed adjustment.

13. The device of claim 11, wherein the determined adjustment includes one of:
a change in an exhalation time; and
a change in a pressure applied by the mechanical ventilator during exhalation.

14. The device of claim 1, further comprising:
an ultrasound imaging device including an ultrasound patch attached to a portion of the patient, wherein the at least one electronic processor controls the ultrasound imaging device to receive the ultrasound imaging data of the diaphragm of the patient from the ultrasound patch; and
a mechanical ventilator configured to deliver mechanical ventilation therapy to the patient.

15. A respiration monitoring method comprising, with an electronic controller:
receiving ultrasound imaging data of a diaphragm of a patient as a function of time during inspiration and expiration while the patient undergoes mechanical ventilation therapy with a mechanical ventilator;
receiving respiratory data of the patient as a function of time during the inspiration and expiration while the patient undergoes the mechanical ventilation therapy;
calculating a positive end-expiratory pressure (PEEP) value for the patient based on the ultrasound imaging data and the respiratory data; and
displaying, on a display device, a representation of the calculated PEEP value.
